(19) United States
(12) Patent Application Publication
(10) Pub. No.: US 2010/0036397 A1
(43) Pub. Date: Feb. 11, 2010

(54) Vascular graft connector

(76) Inventors: Wei-Chang Kang, Tainan (TW); Yu-Li Kang, Tainan (TW)

Correspondence Address: BACON & THOMAS, PLLC, 625 SLATERS LANE, FOURTH FLOOR, ALEXANDRIA, VA, 22314-1176, US

(21) Appl. No.: 12/222,248

(22) Filed: Aug. 6, 2008

(51) Int. Cl. A61B 17/08

(52) U.S. Cl. 606/153

(57) ABSTRACT

The present invention is to provide a prosthetic vascular connector which comprises a male fastener of ring-shaped body and a ring-shaped female fastener; where the outer side of the female fastener is a smooth surface while the inner side, facing the male fastener, is provided with a tapered wall that is gradually contracted inward and an indented catch trough next to the minimum diameter of the tapered wall; when in use, the male fastener and the female fastener each offers the terminal portion of a separate prosthetic blood vessel to pass through its inside, followed by a outward to backward fold around it, and then push the male fastener with the folded terminal portion of the prosthetic blood vessel into the tapered wall of the female fastener covered with the prosthetic blood vessel, where the male fastener is being pushed into a deformed shape to move across the minimum diameter of the tapered wall of the female fastener, and lodged in the catch trough with its original shape restored.

### BACKGROUND OF THE INVENTION

1. Field of the Invention

The present invention relates to prosthetic vascular connectors, more particularly, to one formed by a male fastener and a female fastener, where each offers the terminal portion of a prosthetic blood vessel a penetration through it's inside, followed by an outward to backward fold around it for fastening; the connector features advantages as concise in structure, handy in operation and firm in connection; moreover, there is no contact with the flowing blood for the connector that absolutely avoids thrombus.

2. Description of the Prior Art

The progress of the medical science brings about an abundance of new pharmaceuticals and medical equipment, which prolongs the age of modern human beings considerably. Moreover, the credits are also given to the considerable advancement of expertise of the medical profession and technician. People in the early days realized they were confronted with critical moment of their life when a cardiac surgery was to be operated for curing their heart diseases. The cardiac surgery was for sure risky while the suture of large-vessel by the end of surgery was even more crucial. It's essential to be swift for the suture and to keep on transfusing blood, since it's quite often to bleed during the suture. Even if the surgery is operated smoothly, the observation and treatment after the surgery demand serious caution. It could be fatal to life by any sort of negligence. To surgeons, surgical operations call for superb craft and the capacity to put up with huge pressure, which is indeed a job with injury to body and mind.

Thanks to the advancement of the medical science and technique, many technical aspects of cardiac surgery are now under exceedingly sound control, which could substantially level down the endangerment; especially for the prosthetic blood vessel that can be used as a replacement, which is a real blissful tidings to the cardiovascular patients. However, during the use of a prosthetic blood vessel, there are occasions for how to join the corresponding terminal portions of different prosthetic blood vessels together. The suture was the adopted means in the early days. Since the suture is always progressed at a pretty slow pace and brings about possibility of bleeding, there are inventors having their patents aimed at the above subject matter, for instance: U.S. Pat. No. 6,929,648 and U.S. Pat. No. 6,896,688 are both the patents regarding the connection of prosthetic blood vessels.

Conventional connectors for prosthetic blood vessels have following drawbacks when in use:

(I.) Conventional connectors are made up of a inner ring and an outer binding ring, during using it is essential to have the corresponding terminal portions of different prosthetic blood vessels put around the external surface of the two sides of the inner ring for a preliminary joining, followed by a clamping from the outer binding ring, to achieve a solid joining. Since both the inner ring and the outer binding ring of the connector are rigid body, the terminal portion of a prosthetic blood vessel to be put around the external surface of the inner ring requires a stretch for its terminal portion. This step is hard to operate, and it takes carefulness and patience to accomplish. The success of this capping often requires many times of efforts which deteriorates this joining operation.

(II.) Once a conventional prosthetic vascular connector works for the connection, the flowing blood will keep contacting the internal surface of the inner ring, which causes thrombus effects along the internal surface of the inner ring after a lengthy use, which in turn brings about embolism to the remote blood vessel, causing potential troubles of further processing which again deteriorates the operation.

The inventor recognized the drawbacks of the conventional prosthetic vascular connectors which motivates his further development to achieve a prosthetic vascular connector that features novel in architecture, handy in operation, and excellent in function, which can be of benefit to patients. As a result of this inventive conception, a considerable endeavor of research and development eventually makes the accomplishment of this invention.

### SUMMARY OF THE INVENTION

The objective of the present invention is to provide a prosthetic vascular connector which features concise in architecture, handy in operation, and stable in connection, and no biocompatibility as well.

To achieve the aforesaid objects of the invention, a prosthetic vascular connector of the present invention is made up of a male fastener and a female fastener; where the male fastener is a ring-shaped body; the female fastener is shaped into a ring, where its outer side is a smooth surface while its inner side is provided with a tapered wall that is gradually enlarged outward and an indented catch trough; the tapered wall faces the male fastener, and the maximum diameter of the tapered wall is larger than the outer diameter of the male fastener, while the minimum diameter is smaller than the outer diameter of the male fastener; the catch trough is located next to the minimum diameter of the tapered wall; the male fastener and the female fastener each offers the terminal portion of a prosthetic blood vessel to pass through its inside, followed by a outward to backward fold around it, and then push the male fastener with the folded terminal portion of the prosthetic blood vessel into the tapered wall of the female fastener covered with the prosthetic blood vessel, where the male fastener is being pushed into a deformed shape to move across the minimum diameter of the tapered wall of the female fastener, and lodged in the catch trough with its original shape restored.

### BRIEF DESCRIPTION OF THE DRAWINGS

FIG. 1 is a three-dimensional exploded diagram of the first exemplified embodiment of the present invention which is uncovered by a prosthetic blood vessel;

FIG. 2 is a three-dimensional exploded diagram of the first exemplified embodiment of the present invention which is covered by a prosthetic blood vessel;

FIG. 3 is a sectional side exploded view of the first exemplified embodiment of the present invention which is covered by a prosthetic blood vessel;

FIG. 4 is a sectional side exploded view of the usage of the connection done by the first exemplified embodiment of the present invention;

FIG. 5 is a three-dimensional exploded diagram of the second exemplified embodiment of the present invention; and

FIG. 6 is a sectional side exploded view of the usage of the connection done by the second exemplified embodiment of the present invention.

### DETAILED DESCRIPTION OF THE INVENTION

To achieve the foregoing objects of the present invention, the techniques adopted and the achievable fuction are detailed described with reference to the following preferred exemplified embodiments and the accompanying drawings, which is expected to help those skilled in the art to comprehend the present invention thoroughly.

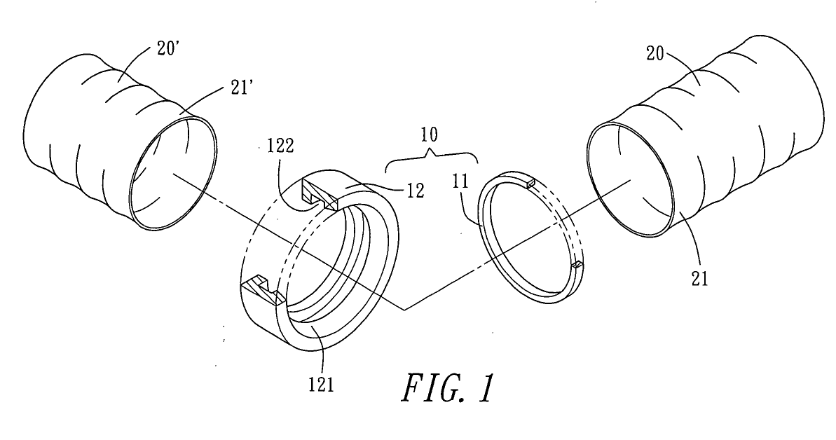

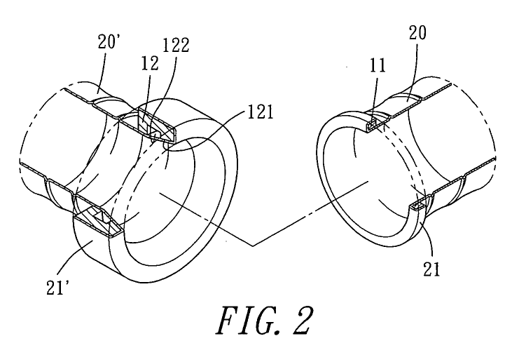

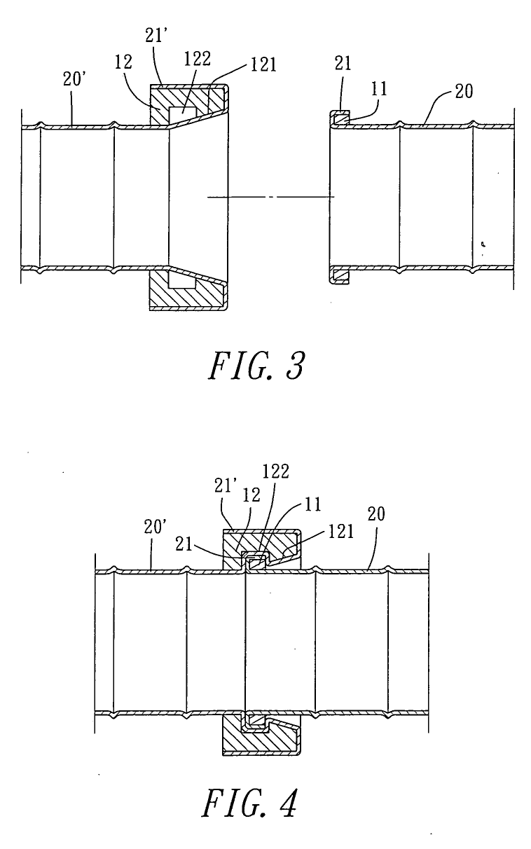

Referring to FIG. 1, the first exemplified embodiment of the present invention is a connector 10, which is used to join the terminal portions 21, 21′ of the two prosthetic blood vessels 20, 20′. The connector 10 is divided architecturally into two components: a male fastener 11 and a female fastener 12, where the male fastener 11 is a ring-shaped body with a inner diameter same as the diameter of the cross section of the terminal portion 21 of a first prosthetic blood vessel 20, which is handy to take the terminal portion 21 of a first prosthetic blood vessel 20 to pass through the inside of the male fastener 11 followed by a reverse fold shown in FIG. 2. The female fastener 12 is shaped into a ring, which is also handy to pass the terminal portion 21′ of a second prosthetic blood vessel 20′ through the inside of the female fastener 12 followed by a reverse fold shown in FIG. 2. The outer side of the female fastener 12 is a smooth surface while the inner side is provided with a tapered wall 121 that is gradually enlarged outward and an indented catch trough 122; When in use, the tapered wall 121 faces the male fastener 11 (shown in FIG. 3), and the maximum diameter (the most outer part) of the tapered wall 121 is larger than the outer diameter of the male fastener 11, so that the male fastener 11 covered with the terminal portion 21 of the first prosthetic blood vessel 20 is still easy to get into the tapered wall 121; where the minimum diameter (the most inner part) of the tapered wall 121 is smaller than the outer diameter of the male fastener 11, so that the male fastener 11 will move hard near the most inner part of the tapered wall 121, at this moment, a bit of inclination for the male fastener 11 will enable part of it to pass the most inner part of the tapered wall 121, followed by a pressing on the male fastener 11 to make it deformed in order to succeeding in passing the rest through the most inner part of the tapered wall 121. Since the male fastener 11 is a ring-shaped body, for the situation of moderate cross section, it can be easily deformed under a pressing, and it restores the deformed body to its original shape when the pressing disappears. Once the male fastener 11 is pushed across the most inner part of the tapered wall 121, it falls into the catch trough 122 for a stay (shown in FIG. 4). Once the inner diameter of the catch trough 122 is designed to be a bit larger than the outer diameter of the male fastener 11, and to be a bit smaller than the net value of the outer diameter of the male fastener 11 plus the thickness of both the first and the second prosthetic blood vessels 20, 20′, then under the status in FIG. 4, the male fastener 11 is in the catch trough 122 with the squeezed vascular walls of the prosthetic blood vessels 20, 20′, which forms a solid and firm connection. It has to note here that the accompanied drawings are done only for description purpose—it is essential that the drawings are enlarged according to the ratio, but in the realization of the invention, the production of the connector is subject to the actual size of the prosthetic blood vessels, to meet the actual requirements.

Accordingly, the connector 10 of the first exemplified embodiment of the invention is designed for a set of components, a male fastener 11 and a female fastener 12, capable of fastening each other, and adopts the way of connection by taking the terminal portions 21, 21′ of the first and the second prosthetic blood vessels 20, 20′ to pass through the inside of the male fastener 11 and the female fastener 12, followed by a reverse fold onto the surfaces of the male fastener 11 and the female fastener 12, and then push the male fastener 11 with the covered first prosthetic blood vessel 20 through the tapered wall 121 of the female fastener 12 covered by the second prosthetic blood vessel 20′ to the catch trough 122, where the male fastener 11 and the vascular walls of the first prosthetic blood vessels 20 and the second prosthetic blood vessels 20′ are altogether put in the catch trough 122 of the female fastener 12.

The connector 10 is an inventive design of the present invention which is used to join prosthetic blood vessels that features the following advantages:
1. First to pass the first and the second prosthetic blood vessels 20, 20′ through the inside of the rings, the male fastener 11 and the female fastener 12, which is easy to accomplish. Then, the terminal portions 21, 21 ′ of the first prosthetic blood vessels 20 and the second prosthetic blood vessels 20′ are reversely folded onto the surfaces of the male fastener 11 and the female fastener 12. This step is also easy to accomplish. Lastly, push the male fastener 11 with the terminal portion 21 of the first prosthetic blood vessel 20 through the tapered wall 121 of the female fastener 12 covered by the second prosthetic blood vessel 20′ to the catch trough 122, which needs an extra pressing. The male fastener 11 is deformed by the pressing but soon restored. Overall, the operation of the invention is quite handy and easy to accomplish.
2. As the male fastener 11 with the covered first prosthetic blood vessels 20 is pushed into the catch trough 122 of the female fastener 12 covered by the second prosthetic blood vessels 20′, the first prosthetic blood vessels 20 and the second prosthetic blood vessels 20′ have already achieved a firm connection, completely free of the possibility of being loosening.
3. When the connection is done for the present invention, all the contact by the flowing blood is merely the internal wall of the prosthetic blood vessel, absolutely no connection with the connector 10; therefore, there won't be thrombus happened at the joining place, nor does it have the danger of causing embolism for the remote blood vessel, completely free of the potential troubles of further processing.

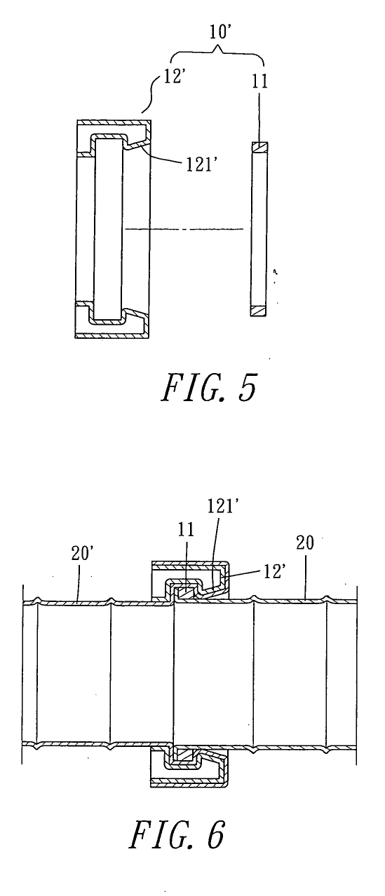

The first exemplified embodiment shown in FIGS. 1-4 shows both the male fastener 11 and the female fastener are solid bodies. In the realization, the female fastener 12 part can be replaced by a bar material with a bend. FIG. 5 shows that the connector 10′ of the second exemplified embodiment is formed by the male fastener 11 and the female fastener 12′, where the male fastener 11 has a moderate thickness, so that it still uses the male fastener 11 as in FIG. 1; the female fastener 12′ has relatively larger thickness so that it cold be better to use the one formed by bending a bar material, which has a sectional side view in FIG. 5. FIG. 6 is a view of the use of the connection between the first prosthetic blood vessel 20 and the second prosthetic blood vessel 20′. The female fastener 12 is formed by bending the bar material, which brings in better elasticity. Once the male fastener 11 with the first prosthetic blood vessel 20 is pushed through the tapered wall 121, the female fastener 12′ is then easier to perform outward stretch, which substantially facilitates the passage of the male fastener 11 with the terminal portion 21 of the first prosthetic blood vessel 20. This is the advantage from the female fastener 12′ made out of the bar material.

In conclusion, the disclosed structure and usage of the present invention have not found in various patents or documents that talk about prior art prosthetic vascular connectors, which is therefore construed to be novel. Moreover, the present invention features extreme handiness in use, which surely achieves the desired objective, and is construed to be creative. Meanwhile, the disclosed content of the present invention is detailed enough to be understood and implemented by those skilled in the art, which is compliant to the complete disclosure, thereby filing the present application herein subject to the patent law, which deserves your favorable examination and approval.

The above disclosed subject matter is merely a preferred exemplified embodiment of the present invention, which is not intended for limiting the scope of the claims of the present invention. Any equivalent variations fall within the real contents and inventive thoughts of the present invention are therefore intended to be embraced by the present invention.

What is claimed is:

1. a prosthetic vascular connector, comprising a male fastener and a female fastener; where said male fastener being a ring-shaped body; said female fastener being shaped into a ring, where its outer side is a smooth surface while its inner side is provided with a tapered wall that is gradually enlarged outward and an indented catch trough; the tapered wall facing said male fastener, and the maximum diameter of the tapered wall being larger than the outer diameter of said male fastener, while the minimum diameter is smaller than the outer diameter of said male fastener; the indented catch trough being located next to the minimum diameter of the tapered wall; said male fastener and said female fastener each offering the terminal portion of a prosthetic blood vessel to pass through its inside, followed by a outward to backward fold around it, and then pushing said male fastener with the folded terminal portion of the prosthetic blood vessel through the tapered wall of said female fastener covered with the prosthetic blood vessel, where said male fastener is being pushed into a deformed shape in order to move across the minimum diameter of the tapered wall of said female fastener, and being lodged in the catch trough with its original shape restored.
